(19) United States
(12) Patent Application Publication
(10) Pub. No.: US 2016/0310108 A1
(43) Pub. Date: Oct. 27, 2016

(54) SOUND DETECTING DEVICE AND METHODS OF USING THE SAME

(71) Applicant: INFRASONIX, INC., Lawrenceville, GA (US)

(72) Inventor: Nigel J. Flynn, Flowery Branch, GA (US)

(21) Appl. No.: 15/140,429

(22) Filed: Apr. 27, 2016

Related U.S. Application Data
(60) Provisional application No. 62/153,146, filed on Apr. 27, 2015.

(51) Int. Cl. A61B 8/08; A61B 7/04; A61B 8/00

(52) U.S. Cl. CPC A61B 8/5207; A61B 8/0883; A61B 8/4209; A61B 8/5223; A61B 7/04

(57) ABSTRACT

Sound detecting devices are disclosed. Methods of using sound detecting devices are also disclosed.

### CROSS-REFERENCE TO RELATED APPLICATIONS

This patent application claims the benefit of priority to U.S. Provisional Patent Application Ser. No. 62/153,146 filed on Apr. 27, 2015 and entitled “SOUND DETECTING DEVICE AND METHODS OF USING THE SAME,” the subject matter of which is hereby incorporated by reference in its entirety.

### FIELD OF THE INVENTION

The present invention is directed to sound detecting devices and methods of using sound detecting devices.

### BACKGROUND

In the United States each year, more than 600,000 people die from cardiovascular disease accounting for one in four mortalities. Of these mortalities, statistics show that some 325,000 people experience their first heart attack and die within approximately one hour of this event due to cardiac arrest. Worldwide these numbers are twenty times larger.

Undiagnosed heart disease is a leading cause of death because no cost-effective technology exists in the hands of every front-line medical professional to identify subjects with either early onset or advanced cardiovascular disease states during an annual check-up. This diagnosis would allow the patient to receive early preventative counseling and recommendations of life-style changes, or in more advanced cases, referral to a cardiologist for timely follow-up care.

Since 1817 medical professionals have relied upon a stethoscope to provide acoustic diagnostic information for medical decisions. The stethoscope continues to provide useful information, but it suffers some limitations. The detection device that the stethoscope relies upon is the human ear, which has the ability to hear sound in a frequency range from approximately 20 to 20,000 hertz. Unfortunately, sound within this frequency range is extensively absorbed by human cellular tissue, which makes it difficult to detect or identify the original source of a given sound.

Recently, a significant breakthrough in sensor technology has allowed scientists for the first time to be able to accurately detect and record sound at frequencies well below the 20 hertz threshold of human hearing, namely “infrasound.” Such a breakthrough in sensor technology was the development of sensors as disclosed in U.S. Pat. No. 8,401,217, the subject matter of which is hereby incorporated by reference in its entirety. Another breakthrough in sensor technology was the development of sensors used in infrasonic stethoscopes as disclosed in U.S. Patent Application Publication Number 2016/0095571, the subject matter of which is hereby incorporated by reference in its entirety.

As evidenced by recent developments in sensor technology, medical diagnostic technology has developed significantly over the last 50 years. Medical diagnostic technology developed over the last 50 years includes, but is not limited to, anatomical methods (such as X-ray Computed Tomography, Computed Tomography Coronary Calcium Score, intima-media thickness [IMT], and intravascular ultrasound [IVUS]), as well as physiological methods (such as lipoprotein analysis, HbA1c, Hs-CRP, and homocysteine), all of which have profoundly influenced both the detection of the onset and treatment of cardiovascular disease. Unfortunately, cardiovascular disease rates and the dramatic mortality rates associated with them have only increased during the last 50-year period.

The anatomical methods directly measure some aspects of the actual process of atherosclerosis itself and therefore offer the possibility of early diagnosis, but these methods are very expensive, involve significant radiation dosages, as in the example of X-ray Computed Tomography (100-1,000 times higher than conventional X-rays—even 5,000 times in the case of multiple uses), or are significantly invasive, as in the case of intravascular ultrasound. The physiological methods are less expensive, but they are not able to quantify the disease state or directly track disease progression. More importantly, existing medical diagnostic technology is unable to achieve the mass proactive monitoring of cardiovascular health that is necessary in order to significantly impact the world's cardiovascular health.

The power of primary proactive monitoring of cardiovascular health is not only that it allows individuals to bring about proactive improvements to their own cardiovascular health through the guidance and assistance of their primary care physicians, but it also permits at-risk patients to be quickly identified, so that they can receive the necessary follow-up attention from a consultant cardiologist, instead of becoming a tragic statistic of mortality from the fourth leading cause of death. This referral will allow a cardiologist to conduct additional follow-up diagnostic work which may include some of the methods outlined earlier, knowing that the high cost and radiation exposure is justified in the context of a defined pre-existing condition, which appropriately justify their use.

For the reasons provided above, efforts continue to further develop medical technology and procedures to further battle cardiovascular disease and other diseases.

### SUMMARY

The present invention addresses some of the difficulties and problems discussed above by the discovery that (1) the human body is a source of infrasound, (2) infrasound is not absorbed by human cellular tissue, and (3) turbulent flow of air or liquid (e.g., within the human body) results in the emission of infrasound. The low frequency of infrasound allows infrasound to pass through tissue without reducing signal amplitude. Initial experimental work, performed in conjunction with the inventors of the infrasonic stethoscope disclosed in U.S. Patent Application Publication Number 2016/0095571, has revealed that extremely clear infrasound signals emerge from the human body.

The sound detecting systems, methods of making sound detecting systems, and methods of using sound detecting devices and systems of the present invention represent technology for providing information for a medical professional diagnostic decision that is:

(1) proactive—the technology will provide information not available today that has the ability to empower patients with this information to control their own health and longevity;

(2) safe—the technology is passive, introducing no radiation or other energy to the body, and can be used multiple times without any health consequences;

(3) non-invasive—the technology does not involve an invasive procedure costing tens of thousands of dollars to diagnose the source of a cardiovascular event; and

(4) inexpensive—the cost of use is expected to be far less per patient than comparable anatomical methods outlined above. Early estimates indicate a 90+% savings to the practitioner which should be reflected in the patient bill.

The disclosed technology does not make the diagnostic methods outlined above (i.e., in the “BACKGROUND” section) irrelevant, but rather makes the above-described diagnostic methods more relevant, as the above-described diagnostic methods will now be able to be used within a justified context of cardiovascular concern raised by the results provided by the device of the present invention.

The present invention has identified a passive detection technology, which is entirely new to medical science. The technology is capable of detecting sound within the frequency range smaller than 0.01 Hz to greater than 20 Hz as it is naturally emitted by the human body. Sound within this frequency range has recently been named “infrasound”. This frequency is outside the range of human hearing which detects sound from 20 Hz to 20,000 Hz.

It has been determined through the use of this technology that the human body is a source of sound within this frequency range. This frequency range provides a rich source of diagnostic information from which a wide range of human disease states can be diagnosed. The technology detects the extent of constricted blood flow in the human cardiovascular system, and provides either early indications of a gradually developing cardiovascular disease states or indicates evidence of an advanced disease state that requires immediate follow-up with a cardiovascular specialist.

This technology has the potential to affordably allow frontline medical practitioners to proactively manage the cardiovascular health of the global population, with the potential to bring about significant global improvements in longevity and morality rates for the world's leading cause of death.

The device (referred to herein as the “INFRASCOPE™ device”) is a medical device that is capable of providing information for medical diagnostic decisions by healthcare professionals. Unlike most diagnostic technologies such as X-Ray Tomography or Echo Ultrasound, the INFRASCOPE™ device introduces no energy into the human body to obtain diagnostic information. The INFRASCOPE™ device simply detects energy emitted passively by the human body using detection technology that is capable of detecting sound within the defined frequency range. The human ear is typically capable of detecting sound at frequencies from 20 to 20,000 hertz. Infrasound is generically defined as those frequencies below that of unaided human ear detection.

The INFRASCOPE™ device produces two types of specific information. First a graphical output identifies specific frequencies of sound associated with identifiable indications. Second, through the use of two or more sensors placed on either side of the anatomy to be investigated, a Doppler effect can be used to create a three-dimensional graphical output. This graphical output allows a medical professional to identify the specific location in which a blockage within the vascular system has taken place.

The INFRASCOPE™ device technology can be used to diagnose a wide range of additional disease states beyond cardiovascular disease.

As discussed above, recent breakthroughs in sensor technology now allows scientists for the first time to be able to accurately detect and record “infrasound” at frequencies well below the 20 hertz threshold of human hearing. The present invention utilizes this recent discovery by measuring a key source of infrasound, namely, turbulent flow induced in blood when passing through the cardiovascular system of an animal. The present invention has discovered that an increase in arthrosclerosis within the cardiovascular system results in increasingly turbulent blood flow, and the measurable change and increase in the quantity of infrasound emitted by the human cardiovascular system is measurable by the INFRASCOPE™ device. Accordingly, the INFRASCOPE™ device enables the extent of cardiovascular disease within the human body to be quantified, so that an individual is able to receive a very precise output, e.g., expressed as a number between 1 and 100, which communicates their cardiovascular health state via the INFRASCOPE™ device.

In addition, the INFRASCOPE™ device provides a visual three-dimensional image of the sound as it emerges from the human body. Today, ultrasound images of the human body are capable of being created from ultrasound introduced into the human body from an ultrasound device. The absorbed and reflected ultrasound is then gathered in a detection device to create an ultrasound image. Even in the most sophisticated systems, the image lacks definition, appearing blurred and difficult to interpret.

By contrast, infrasound used by the INFRASCOPE™ device is not absorbed by human tissue. Consequently, passive human infrasound provides precise sound-source images of the human body via the INFRASCOPE™ device, which is able to provide comprehensive images that indicate areas of constricted cardiovascular blood flow.

Accordingly, the present invention is directed to sound detecting systems. In one exemplary embodiment, the sound detecting device of the present invention comprises: one or more sensors, each sensor being capable of detecting infrasound produced by the human body and in a frequency range of less than about 30 hertz (Hz). In some embodiments, the sound detecting device of the present invention comprises: two or more sensors, each sensor being capable of detecting infrasound produced by the human body in a frequency range of less than about 30 hertz (Hz).

The present invention is further directed to use of the disclosed sound detecting device. In one exemplary embodiment, the use of the disclosed device comprises using a device to detect infrasound from within the human body, the device comprising: one or more sensors, each sensor being capable of detecting infrasound produced by the human body in a frequency range of less than about 30 hertz (Hz).

The present invention is even further directed to methods of using the disclosed sound detecting device. In one exemplary embodiment, the method of using the disclosed sound detecting device comprises positioning the device along an outer surface of the human body, wherein the device comprises: one or more sensors, each sensor being capable of detecting infrasound produced by the human body in a frequency range of less than about 30 hertz (Hz).

These and other features and advantages of the present invention will become apparent after a review of the following detailed description of the disclosed embodiments and the appended claims.

### BRIEF DESCRIPTION OF THE DRAWINGS

FIG. 1 is a perspective view of an exemplary sound detection system of the present invention;

FIG. 2 is a cross-sectional view of an exemplary sensor suitable for use in the exemplary sound detection system shown in FIG. 1;

FIG. 3 is a flow diagram of electronics suitable for use in the exemplary sound detection system shown in FIG. 1 so as to process signals from the sensors used in the exemplary sound detection system of the present invention; and

FIG. 4 is a flow diagram of electronics/software suitable for use in the exemplary sound detection system shown in FIG. 1 so as to process signals from the sensors and generate three dimensional images for display.

### DETAILED DESCRIPTION

To promote an understanding of the principles of the present invention, descriptions of specific embodiments of the invention follow and specific language is used to describe the specific embodiments. It will nevertheless be understood that no limitation of the scope of the invention is intended by the use of specific language. Alterations, further modifications, and such further applications of the principles of the present invention discussed are contemplated as would normally occur to one ordinarily skilled in the art to which the invention pertains.

The present invention is directed to sound detecting devices and methods of using the same. The disclosed INFRASCOPE™ device provides early diagnostic information concerning the development of cardiovascular disease (CVD), and identifies subjects (e.g., patients) with either early onset or advanced disease states. These patients can then receive appropriate follow-up care, avoid serious CVD-related events, and extend their lives through proactive medical care.

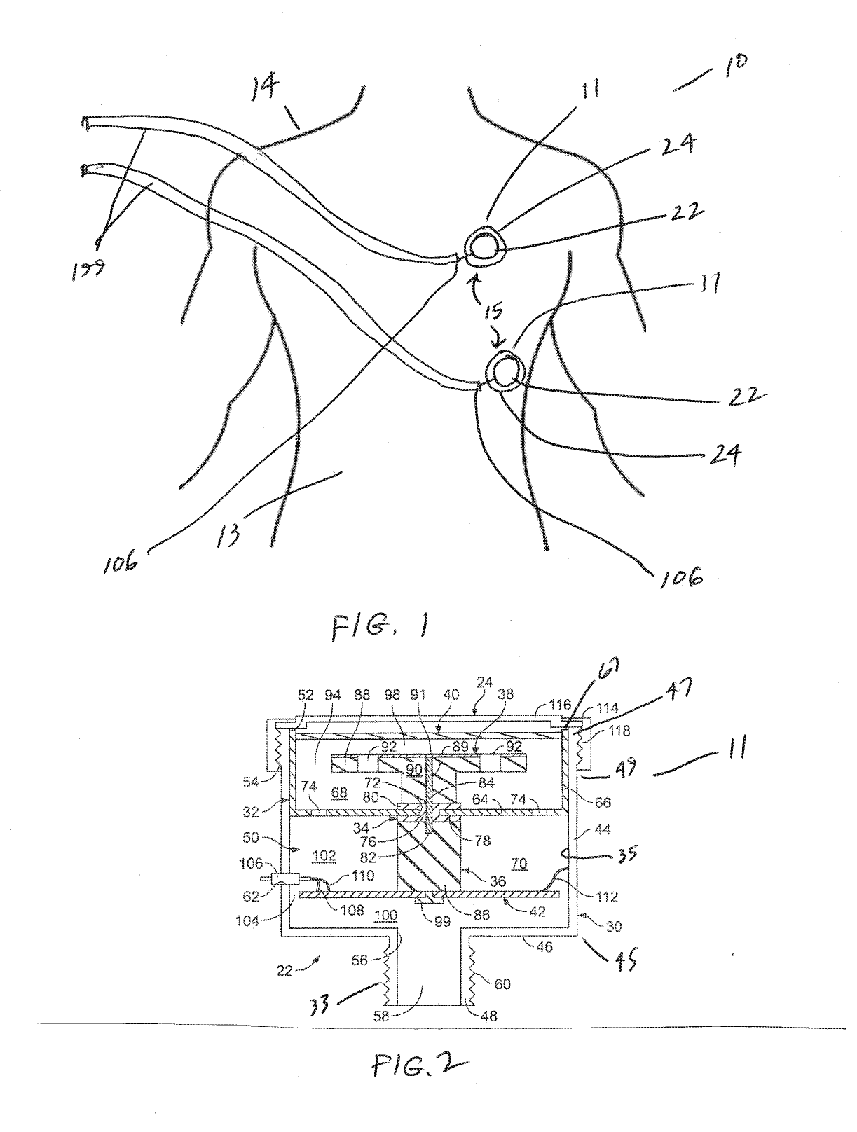

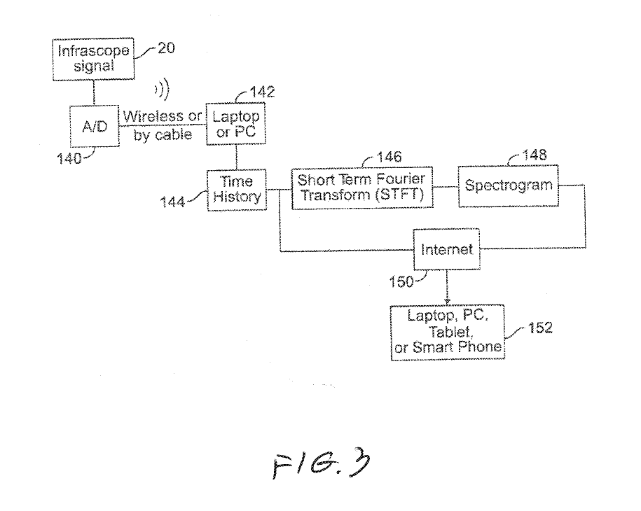

As shown in FIG. 1, an exemplary sound detection system 10 of the present invention comprises two sensors 11 positioned along an outer surface 13 of patient 14. It should be understood that a given sound detection system of the present invention may comprise one or more sensors 11 positioned proximate to any outer surface 13 of patient 14 (e.g., one on a front chest surface and another on a rear chest/back surface). Each sensor 11 is further described with reference to FIG. 2.

As described in U.S. Patent Application Publication Number 2016/0095571 and shown in FIG. 2, each exemplary sensor 11 may comprise microphone 22, a cup-like body 30, a cup-like support plate 32, an insulating member 34, a conductor 36, a backplate 38, a membrane 40 and a low-noise preamplifier board 42. Body 30 has a cylindrical side wall 44 having a proximal end 45 and a distal end 47, an end wall 46 at proximal end 45 of body 30, and a connection port 48 extending proximally from end wall 46. Body 30 may be formed of metal, such as a stainless steel or aluminum. Side wall 44 and end wall 46 define an internal cavity 50 within body 30. Distal end 47 of body 30 is open such that an aperture 52 is defined in body 30. A thread form 54 is provided on the exterior surface 49 of side wall 44 at distal end 47.

End wall 46 substantially closes proximal end 45 of body 30 with the exception of an aperture 56 therethrough, and may extend perpendicularly relative to side wall 44. Aperture 56 may be centrally located in end wall 46 and is within connection port 48. Connection port 48 extends proximally from end wall 46 and has a passageway 58 therethrough, which is in communication with cavity 50 via aperture 56. Exterior surface 33 of connection port 48 has a thread form 60 thereon. An aperture 62 is provided through side wall 44 at a position spaced from proximal end 45 of side wall 44.

Support plate 32 is attached to an internal surface 35 of side wall 44 and seats within cavity 50. Support plate 32 may be formed of metal, and has a circular base wall 64, which spans the diameter of side wall 44 and is parallel to end wall 46, and a depending side wall 66, which extends distally from base wall 64. Side wall 66 terminates in a free end 67. Side wall 66 engages against internal surface 35 of side wall 44 of body 30, such that free end 67 of side wall 66 is proximate to distal end 47 of body 30, and base wall 64 is spaced from distal end 47 of body 30. Support plate 32 is affixed to body 30 by suitable means, such as welding, in such a way that whole assembly can be connected to the ground of preamplifier board 42. As a result of this arrangement, a distal chamber 68 is formed between base wall 64 and distal end 47 of body 30, and a proximal chamber 70 is formed between base wall 64 and proximal end 45 of body 30. Base wall 64 has an aperture 72 therethrough, which may be centrally located. Base wall 64 also has at least one aperture 74 or slot therethrough to allow air to flow from distal chamber 68 to proximal chamber 70.

The insulating member 34, which may be formed of plastic, ceramic, wood or any suitable insulating material, seats within aperture 72 in support plate 32 and is used to electrically isolate conductor 36, backplate 38 and preamplifier board 42 from support plate 32. As shown, insulating member 34 has a central portion 76, which extends through aperture 72, a proximal portion 78, which extends radially outwardly from central portion 76 on the distal side of base wall 64, and a distal portion 80, which extends radially outwardly from central portion 76 on the proximal side of base wall 64. A passageway 82 extends through central portion 76.

Backplate 38 is formed of a conducting material, and is formed from a base wall 88 and may further be formed of a proximal extending portion 90, which extends perpendicularly from base wall 88. Backplate 38 may be formed of, for example, from conducting ceramics, brass, or stainless steel. A passageway 89 extends through base wall 88, and extending portion 90 if provided, from its proximal surface to its distal surface. A permanently polarized thin polymer film 91 is coated on the distal surface of backplate 38. Polarized thin polymer film 91 operates without the need for an external power supply. As described in U.S. Pat. No. 8,401,217, the subject matter of which is hereby incorporated by reference in its entirety, backplate 38 has a plurality of spaced apart holes 92 therethrough (two holes are visible in FIG. 2). Extending portion 90 engages against distal portion 80 of insulating member 34, and is secured to a distal end of conductor 36, such that backplate 38 and conductor 36 are in electrical communication. Base wall 88 of backplate 38 is parallel to base wall 64 of support plate 32. A slot 94 is defined between the outer diameter of backplate 38 and side wall 44 of body 30. The area between backplate 38 and the proximal end 45 of body 30 defines a backchamber.

Conductor 36 extends through passageways 82, 89 and extends into proximal chamber 70. Conductor 36 is electrically connected to backplate 38. As shown, conductor 36 is formed of a conducting rod or wire 84, which extends through passageways 82, 89, and a conductive rod 86 extending proximally from conducting rod or wire 84 and insulating member 34. If formed of two components, the components are suitably connected to each other to form an electrical connection. Rod or wire 84 and rod 86 may be formed of brass, or may be formed of differing conductive materials. The proximal end of conductor 46 is proximate, to but spaced from, end wall 46 such that a gap is defined therebetween.

Membrane 40 is formed of a flexible conductive material and is seated at distal free end 67 of side wall 66 of support plate 32 such that the membrane 40 is positioned within distal chamber 68 and is proximate to, but spaced from, distal end 47 of body 30. The diameter of membrane 40 is selected so that membrane 40 stays within side wall 66. Membrane 40 is parallel to end wall 46 of body 30 and to base wall 64 of support plate 32. As a result, membrane 40 is in electrical communication with support plate 32. The tension of membrane 40 may be less than about 400 Newton per meter.

Backplate 38 is proximate to, but spaced from membrane 40, such that an air gap 98 is formed between membrane 40 and backplate 38 to create a capacitor in microphone 22 as is described in U.S. Pat. No. 8,401,217. As described in U.S. Pat. No. 8,401,217, the number, locations and sizes of holes 92, the size of slot 94, and the inner volume of the backchamber are selected such to allow enough air flow to provide proper damping of the motion of membrane 40. As described in U.S. Pat. No. 8,401,217, the backchamber serves as a reservoir for the airflow through holes 92 in backplate 38.

As described in U.S. Patent Application Publication Number 2016/0095571, in an exemplary embodiment, membrane 40 has a diameter of approximately 1.05 inches (0.0268 meter). Membrane 40 may have the following characteristics/dimensions: radius=0.0134 meter; thickness=2.54×10⁻⁵ meter; density=8000 kilogram/meter³; tension=400 N/meter; surface density=0.1780 kilogram/meter²; and stress=47.4045 PSI. Further, microphone 22 may comprises an air layer, which may have the following characteristics/dimensions: air gap=2.54×10⁻⁵ meter; density=1.2050 kilogram/meter³; viscosity=1.8×10⁻⁵ Pascal-second; sound velocity through the air gap=290.2 meters per second; and gamma=1.4. Microphone 22 may also comprise a slot 94, which may have the following characteristics/dimensions: distance from the center of the backplate=0.0117 meter; width=0.00351 meter; depth=0.00114 meter; and area=0.000258 meter². Backplate 38 may define six holes 92, and each hole 92 may have the following characteristics/dimensions: distance from center of backplate to center of hole=0.00526 meter; radius=0.002 meter; depth=0.045 meter; angle between two lines going from center of backplate to either side edge of hole=43.5 degrees; and area=1.26×10⁻⁵ meter². Microphone 22 may also have the following further characteristics/dimensions: volume of the backchamber=5×10⁻⁵ meter³; membrane mass=480 kilogram/meter²; membrane compliance=3.2×10⁻¹¹ meter⁵/Newton; and air gap compliance=3.5×10⁻¹⁰ meter⁵/Newton. In one exemplary embodiment, the resonant frequency of microphone 22 may be 3108.01 Hertz.

Preamplifier board 42 is planar and extends radially outwardly from the proximal end of conductor 36. Preamplifier board 42 is connected to the proximal end of conductor 36 by suitable means such that there is an electrical connection between preamplifier board 42 and conductor 36, such as a brass screw 99. Preamplifier board 42 is parallel to end wall 36 of body 30, base wall 64 of support plate 32 and base wall 88 of backplate 38. The position of preamplifier board 42 defines a first proximal chamber 100, which has a volume V1 between preamplifier board 42 and end wall 46 of body 30, and a second distal chamber 102, which has a volume V2 between preamplifier board 42 and base wall 64 of support plate 32. A slot 104 is defined between the outer diameter of preamplifier board 42 and side wall 44 of body 30 to allow air to flow from distal chamber 102 to proximal chamber 100. In one embodiment, volume V1 is approximately 0.1287 cubic inch, and volume V2 is approximately 0.6 cubic inch. The air can only flow from distal chamber 102 to proximal chamber 100 through slot 104. In one embodiment, slot 104 has a clearance distance between the outer diameter of preamplifier board 42 and side wall 44 of approximately 0.025″, which slot 104 extends around preamplifier board 42. An electrical connection 106 extends through aperture 62 in side wall 44 and is sealed to side wall 44 by suitable means. Electrical connection 106 is electrical communication with preamplifier board 42 via wires 108, 110. Preamplifier board 42 is also electrically connected to body 30 via a wire 110, which provides a ground to preamplifier board 42. Preamplifier board 42 contains known components for measuring the capacitance between membrane 40 and backplate 38, and converting this measured capacitance into voltage.

Connection port 48 may be connected to a distal end of a flexible tube (i.e., such as flexible tube 26 shown in U.S. Patent Application Publication Number 2016/0095571), which may be formed of latex or rubber, and which has an earpiece (i.e., such as earpiece 28 shown in U.S. Patent Application Publication Number 2016/0095571) at the proximal end of the tube. Such a flexible tube and earpiece, like a typical stethoscope, are known in art for transmitting sound. The flexible tube, when present, is attached to connection port 48, such that there is no air exchange between the flexible tube and body 30, and such that the passageway through the tube is in communication with distal chamber 100 via passageway 58 and aperture 56. When the earpiece is inserted into the ears of the medical personnel, this allows substantially no air exchange between cavity 50 of microphone 22 and the outside of microphone 22. The length of the flexible tube is adjusted so that maximum audible sound is received at the earpiece, which are used by medical personnel to hear the desired sounds in real time.

In other embodiments, a cap (not shown) may be positioned over connection port 48 to seal this opening of body 30. In yet another embodiment, connection port 48 is not present, and end wall 46 of body 30 is continuous (i.e., there are no apertures/opening within or thru end wall 46).

The combination of volumes V1 and V2 and slot 104 around preamplifier board 42 provide sufficient acoustic resistance for pressure equalization, and lowers the low frequency threshold. When a flexible tube is connected to an earpiece, due to increased acoustic resistance and longer required period for pressure equalization, this lowers the low −3 dB frequency to 0.03 Hertz.

As described in U.S. Patent Application Publication Number 2016/0095571, the microphone may differ from U.S. Pat. No. 8,401,217 in that preamplifier board 42 is mounted horizontally in body 30 to divide the backchamber into two lower chambers 100 and 102 and that preamplifier board 42 is parallel to membrane 40, rather than being positioned vertically that is perpendicular to membrane 40 as is positioned in U.S. Pat. No. 8,401,217, and in that the grid of U.S. Pat. No. 8,401,217 is eliminated and instead body 30 includes threads 54 for connection of body coupler 24 (or body coupler 24a as discussed in U.S. Patent Application Publication Number 2016/0095571) to distal end 47 of body 30.

Body coupler 24 (or body coupler 24a) threadedly attaches to thread form 54 at distal end 47 of body 30 such that there is no air exchange between body coupler 24 (or body coupler 24a) and body 30. In one embodiment, as shown in FIG. 2, body coupler 24 is formed of an outer ring 114, which has a flexible non-conductive diaphragm 116 attached thereto and which spans the diameter of ring 114. Outer ring 114 may be formed either of thermoplastic polyurethane elastomers (TPU) or of closed cell polyurethane foam material, which can be made of different densities, and has an internal thread form 118 for attachment of outer ring 114 to distal end 47 of body 30. The TPU material is used when full spectrum of acoustic signals are to be recorded from a heart and closed cell polyurethane foam material is used only when infrasonic signals is to be recorded as this material acts as a passive filter and audible sound is shunted off. When attached, membrane 40 of microphone 22 and diaphragm 116 of body coupler 24 (or body coupler 24a) is approximately 0.1 inch apart. Body coupler 24 (or body coupler 24a) is placed against the body of the patient during the monitoring of the physiological process.

In some embodiments, a body coupler 24a as shown in FIG. 5A of U.S. Patent Application Publication Number 2016/0095571 may be used in sound detecting systems of the present invention and the sensor 11 used therein. However, in preferred embodiments of the present invention, a body coupler such as body coupler 24 is used in a non-invasive method of detecting infrasound of a patient (e.g., patient 14 shown in FIG. 1).

As discussed herein, preamplifier board 42 is installed parallel to base wall 54 and to membrane 24. Slot 104 between the edge of preamplifier board 42 and side wall 44 is small, for example 0.025″, to increase acoustic resistance. The combined volumes V1 and V2 and the volume in the flexible tube, when present, is less than or equal to about 5×10⁻⁵ meter³. Because of increased acoustic resistance, pressure equalization takes longer, which aids in lower −3 dB frequency to 0.03 Hertz.

As shown in the block diagram of FIG. 3, in some embodiments, signals from sensors 11 may be digitized via an analog to digital digitizer board 140. Once digitized, the signal is transmitted wirelessly or by cable to workstation 142, such as a laptop or personal computer. At 144, time history is plotted for data collected at different locations of patient 14 such as at locations 15 shown in FIG. 1. Workstation 142 provides control, analysis and display of the recorded data. MATLAB software may also be used to process the data to generate real-time spectrograms using short-time Fourier transform (STFT) spectrum of the corresponding data at 146 and 148. The time history and spectrogram of biological signals is transferred by the Internet 150 to a remote workstation 152, if desired, for observation and analysis. Examples of such remote workstations 152 may be a remote computer monitor, smartphone or tablet. The signals may be sent via wired connection, or may be wirelessly transmitted, such as by using commercially available Bluetooth module, to PC or laptop for processing. The data is converted in useful visual format also called spectrogram, which may be helpful for physician to diagnose any abnormality. The display of short term spectra is performed in real time, in order to detect the presence of a short term event in the data.

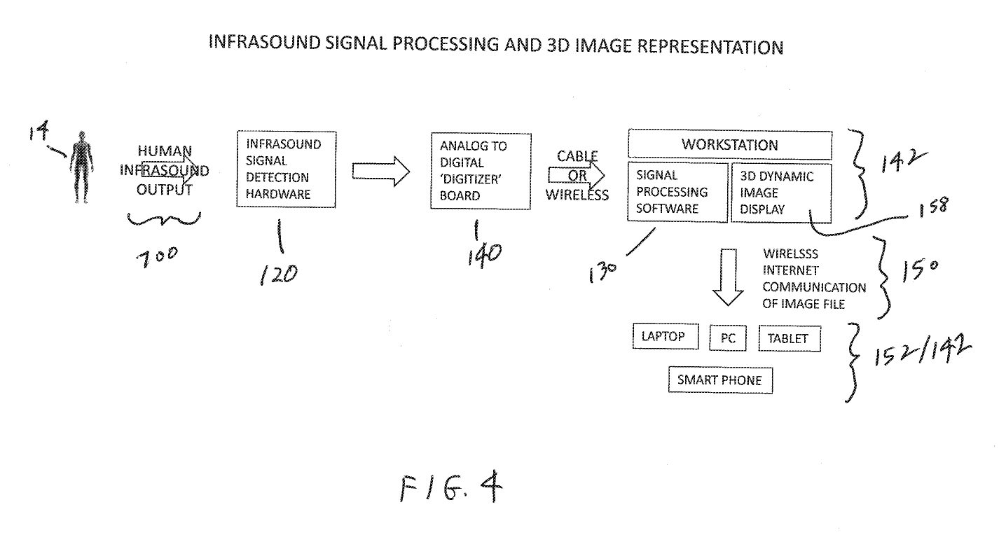

As shown in the block diagram of FIG. 4, in some embodiments, signals 700 from sensors 11 may be (i) detected using infrasound signal detection hardware 120 (e.g., the device as described in U.S. Pat. No. 8,401,217, namely, sensors and integrated pre-amplification board), (ii) digitized via an analog to digital digitizer board 140, and (iii) transmitted wirelessly or by cable to one or more workstations 142, such as a laptop or personal computer, and (iv) converted into one or more image files. The one or more image files may be subsequently transmitted wirelessly or by cable to one or more workstations 142 and/or one or more remote workstations 152, if desired, for observation and analysis. In some embodiments, the step of converting signals 700 from sensors 11 into one or more image files may be performed via signal processing software 130 (e.g., any number of commercially available three-dimensional image processing software packages) so as to generate three dimensional (3D) images, which may be displayed on a 3D dynamic image display 158.

### Other Embodiments

Sound Detecting Devices and Systems
1. A sound detecting device comprising: one or more sensors, each sensor being capable of detecting infrasound produced by the human body and in a frequency range of less than about 30 hertz (Hz). In some embodiments, each sensor comprises a sensor similar to those used in the infrasonic stethoscope 20 disclosed in U.S. Patent Application Publication Number 2016/0095571. In other embodiments, each sensor comprises a sensor similar to those used in the infrasonic stethoscope 20 disclosed in U.S. Patent Application Publication Number 2016/0095571 except end wall 46 is continuous without opening 58 and port 48. Each sensor 11 comprises, inter alia, microphone 22, a body coupler 24 or 24a attached to microphone 22, and the other sensor components discussed above. Along with each sensor, a given sound detecting device (or system) may further comprise electronics used to convert a signal from the sensor (i.e., sensor 11) into, for example, a three-dimensional image.
2. The sound detecting device of embodiment 1, wherein the frequency range is greater than 0 Hz to about 20 Hz.
3. The sound detecting device of embodiment 1 or 2, wherein the device is capable of detecting infrasound output from the human body as a result of energy introduced into the human body from an external source other than the sound detecting device.
4. The device of any one of embodiments 1 to 3, wherein the device is capable of detecting infrasound by placing the device proximate the human body.
5. The device of any one of embodiments 1 to 4, wherein the device is capable of detecting infrasound by placing the device on a garment worn by the human body.
6. The device of any one of embodiments 1 to 5, wherein the device provides digital output as a function of one or more of: energy intensity, energy frequency and time.
7. The device of any one of embodiments 1 to 6, wherein the device provides output that can be correlated to individual physical structures within the human body.
8. The device of any one of embodiments 1 to 7, wherein the device provides cumulative energy output from one or more cardiovascular related structures of the human body, the cumulative energy output added together providing a quantification of a total cardiovascular disease state of a given subject.
9. The device of any one of embodiments 1 to 8, wherein the device is capable of collecting infrasound information within thirty (30) seconds or less, the infrasound information being sufficient to provide comprehensive cardiovascular disease state quantification and other important information for disease state diagnosis.
10. The device of any one of embodiments 1 to 9, wherein the device is capable of (i) identifying a source of infrasound from within the human body, and (ii) providing disease diagnosis based upon infrasound output from a wide range of biological structures within the human body.
11. The device of any one of embodiments 1 to 10, wherein the device comprising two or more sensors.
12. The device of embodiment 11, wherein the two or more sensors are located in close proximity to a skin surface of the human body and placed in such a way that a coordinated digital output from the two or more sensors is renderable as a three dimensional image via a three dimensional image generator. For example, the three dimensional image generator may comprise (i) any device (or combination of devices) capable of receiving the coordinated digital output from the two or more sensors and converting the coordinated digital output into three dimensional images, (ii) three dimensional image generating software, (iii) an analog to digital signal converter, (iv) a laptop (or other computer) capable of running the three dimensional image generating software, or (v) any combination of (i) to (iv).
13. The device of embodiment 11 or 12, wherein the two or more sensors are located on a ventral and a dorsal surface of the human body.
14. The device of embodiment 12 or 13, wherein the three dimensional image indicates locations of high energy intensity as a function of energy frequency and time.
15. The device of any one of embodiments 12 to 14, wherein the three dimensional image identifies individual physical structures within the human body.
16. The device of any one of embodiments 12 to 15, wherein the device provides cumulative energy output from one or more cardiovascular related structures of the human body, the cumulative energy output added together providing a quantification of a total cardiovascular disease state of a given subject.
17. The device of any one of embodiments 12 to 16, wherein the device is capable of collecting infrasound information within thirty (30) seconds or less, the infrasound information being sufficient to provide comprehensive cardiovascular disease state quantification and other important information for disease state diagnosis.
18. The device of any one of embodiments 12 to 17, wherein the device is capable of (i) identifying a source of infrasound from within the human body, and (ii) providing disease diagnosis based upon infrasound output from a wide range of biological structures within the human body.

Use of the Sound Detecting Devices and Systems:
19. Use of the device of any one of embodiments 1 to 18 so as to detect infrasound from within the human body.

Methods of Using the Sound Detecting Devices and Systems:
20. A method of using the device of any one of embodiments 1 to 18, said method comprising: positioning the device along an outer surface of the human body.
21. The method of embodiment 20, wherein each sensor is capable of detecting infrasound produced by the human body in a frequency range of greater than 0 Hz to about 20 Hz.
22. The method of embodiment 20 or 21, further comprising: introducing energy into the human body from an external source other than the sound detecting device; and detecting infrasound output from the human body as a result of said introducing step.
23. The method of any one of embodiments 20 to 22, wherein said positioning step comprises placing the device on a garment worn by the human body.
24. The method of any one of embodiments 20 to 23, further comprising: collecting digital output from the sound detecting device as a function of one or more of: energy intensity, energy frequency and time.
25. The method of any one of embodiments 20 to 24, further comprising: correlating digital output from the sound detecting device to individual physical structures within the human body.
26. The method of any one of embodiments 20 to 25, further comprising: providing a quantification of a total cardiovascular disease state of a given subject to the subject, the total cardiovascular disease state being derived from cumulative energy output from one or more cardiovascular related structures of the human body as measured by the device.
27. The method of any one of embodiments 20 to 26, further comprising: collecting infrasound information from the device within thirty (30) seconds or less, the infrasound information being sufficient to provide comprehensive cardiovascular disease state quantification and other important information for disease state diagnosis.
28. The method of any one of embodiments 20 to 27, further comprising: identifying a source of infrasound from within the human body, and providing disease diagnosis based upon infrasound output from a wide range of biological structures within the human body.
29. The method of any one of embodiments 20 to 28, further comprising: positioning two or more sensors of the device along an outer surface of the human body.
30. The method of embodiment 29, wherein the two or more sensors are positioned in close proximity to a skin surface of the human body and placed in such a way that a coordinated digital output from the two or more sensors is renderable as a three dimensional image.
31. The method of embodiment 29 or 30, wherein the two or more sensors are located on a ventral and dorsal surface of the human body.
32. The method of embodiment 30 or 31, wherein the three dimensional image indicates locations of high energy intensity as a function of energy frequency and time.
33. The method of any one of embodiments 30 to 32, wherein the three dimensional image identifies individual physical structures within the human body.
34. The method of any one of embodiments 30 to 33, wherein the device provides cumulative energy output from one or more cardiovascular related structures of the human body, the cumulative energy output added together providing a quantification of a total cardiovascular disease state of a given subject.
35. The method of any one of embodiments 30 to 34, wherein the device is capable of collecting infrasound information within thirty (30) seconds or less, the infrasound information being sufficient to provide comprehensive cardiovascular disease state quantification and other important information for disease state diagnosis.
36. The method of any one of embodiments 30 to 35, wherein the device is capable of (i) identifying a source of infrasound from within the human body, and (ii) providing disease diagnosis based upon infrasound output from a wide range of biological structures within the human body.

It should be understood that although the above-described sound detecting devices, systems, and methods are described as “comprising” one or more components or steps, the above-described sound detecting devices, systems, and methods may “comprise,” “consists of,” or “consist essentially of” any of the above-described components or steps of the sound detecting devices, systems, and methods. Consequently, where the present invention, or a portion thereof, has been described with an open-ended term such as “comprising,” it should be readily understood that (unless otherwise stated) the description of the present invention, or the portion thereof, should also be interpreted to describe the present invention, or a portion thereof, using the terms “consisting essentially of” or “consisting of or variations thereof” as discussed below.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a sound detecting device, system, and/or method that “comprises” a list of elements (e.g., components or steps) is not necessarily limited to only those elements (or components or steps), but may include other elements (or components or steps) not expressly listed or inherent to the sound detecting device, system, and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a sound detecting device, system, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

Sound detecting devices were prepared and utilized to measure infrasound of the human body.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A sound detecting device comprising:
two or more sensors, each sensor being capable of detecting infrasound produced by the human body in a frequency range of less than about 30 hertz (Hz); and
a three dimensional image generator in communication with said two or more sensors, said two or more sensors being located in close proximity to a skin surface of the human body and placed in such a way that a coordinated digital output from the two or more sensors is renderable as a three dimensional image via said three dimensional image generator.

2. The sound detecting device of claim 1, wherein the frequency range is greater than 0 Hz to about 20 Hz.

3. The sound detecting device of claim 1, wherein the device is capable of detecting infrasound output from the human body as a result of energy introduced into the human body from an external source other than the sound detecting device.

4. The device of claim 1, wherein the device provides output that can be correlated to individual physical structures within the human body.

5. The device of claim 1, wherein the device is capable of collecting infrasound information within thirty (30) seconds or less, the infrasound information being sufficient to provide comprehensive cardiovascular disease state quantification and other important information for disease state diagnosis.

6. The device of claim 1, wherein the two or more sensors are located on a ventral and dorsal surface of the human body.

7. The device of claim 1, wherein the three dimensional image indicates locations of high energy intensity as a function of energy frequency and time.

8. The device of claim 1, wherein the three dimensional image identifies individual physical structures within the human body.

9. The device of claim 1, wherein the device provides cumulative energy output from one or more cardiovascular related structures of the human body, the cumulative energy output added together providing a quantification of a total cardiovascular disease state of a given subject.

10. The device of claim 1, wherein the device is capable of collecting infrasound information within thirty (30) seconds or less, the infrasound information being sufficient to provide comprehensive cardiovascular disease state quantification and other important information for disease state diagnosis.

11. The device of claim 1, wherein the device is capable of (i) identifying a source of infrasound from within the human body, and (ii) providing disease diagnosis based upon infrasound output from a wide range of biological structures within the human body.

12. A method of using the device of claim 1, said method comprising:
positioning the device along an outer surface of the human body.

13. The method of claim 12, wherein each sensor is capable of detecting infrasound produced by the human body in a frequency range of greater than 0 Hz to about 20 Hz.

14. The method of claim 12, further comprising:
introducing energy into the human body from an external source other than the sound detecting device; and
detecting infrasound output from the human body as a result of said introducing step.

15. The method of claim 12, wherein said positioning step comprises placing the device on a garment worn by the human body.

16. The method of claim 12, further comprising:
collecting digital output from the sound detecting device as a function of one or more of: energy intensity, energy frequency and time.

17. The method of claim 12, wherein the two or more sensors are located on a ventral and dorsal surface of the human body.

18. The method of claim 12, wherein the device provides cumulative energy output from one or more cardiovascular related structures of the human body, the cumulative energy output added together providing a quantification of a total cardiovascular disease state of a given subject.

19. The method of claim 12, wherein the device is capable of collecting infrasound information within thirty (30) seconds or less, the infrasound information being sufficient to provide comprehensive cardiovascular disease state quantification and other important information for disease state diagnosis.

20. The method of claim 12, wherein the device is capable of (i) identifying a source of infrasound from within the human body, and (ii) providing disease diagnosis based upon infrasound output from a wide range of biological structures within the human body.
